(19) United States
(12) Patent Application Publication
(10) Pub. No.: US 2019/0374808 A1
(43) Pub. Date: Dec. 12, 2019

(54) FLYWHEEL MEASURING SYSTEM AND FITNESS EQUIPMENT HAVING THE SAME

(71) Applicant: TUNG KENG ENTERPRISE CO., LTD., Taichung City (TW)

(72) Inventor: Chia-Yen HUANG, Taichung City (TW)

(73) Assignee: TUNG KENG ENTERPRISE CO., LTD., Taichung City (TW)

(21) Appl. No.: 16/004,079

(22) Filed: Jun. 8, 2018

(51) Int. Cl. A63B 21/22; H02K 7/02; A61B 5/22; A63B 21/005; A63B 21/00; A63B 22/06; A63B 24/00

(52) U.S. Cl. CPC A63B 21/225; H02K 7/025; A61B 5/221; A61B 5/224; A63B 21/0052; A63B 2024/0065; A63B 22/0605; A63B 24/0062; A61B 2562/0261; A63B 2220/833; A63B 21/4034

(57) ABSTRACT

A flywheel measuring system includes a base, a flywheel, at least one permanent magnet, a magnetic core, an abutting member fixed on the base, a cantilever arm, and a signal generating apparatus. The flywheel is rotatably disposed on the base and is provided with at least one permanent magnet. The magnetic core is movably disposed on the base and is electromagnetically coupled to the at least one permanent magnet. The cantilever arm is disposed on the base. The signal generating apparatus is disposed on the cantilever arm. When the flywheel rotates, the at least one permanent magnet drives the magnetic core to move, and the magnetic core pushes the cantilever arm to approach the abutting member, so that the signal generating apparatus generates a strain and outputs a signal. The fitness equipment could calculate an output power when a rider pedals the pedal according to the signal.

### BACKGROUND OF THE INVENTION

### Technical Field

The present invention relates generally to a flywheel measuring system and a fitness equipment, and more particularly to a flywheel measuring system and a fitness equipment which could measure a rider's pedaling power.

### Description of Related Art

Conventional fitness equipment, such as a treadmill, an elliptical trainer, and a stationary bicycle, is commonly used to improve cardiopulmonary function, wherein many people choose an elliptical trainer and stationary bicycle for aerobic exercise since elliptical trainer and stationary bicycle have less injury to the user's knee during exercise.

In order to allow users to adjust an exercise force by themselves, a magnetic resistance device is disposed inside of elliptical trainer and stationary bicycle. For instance, U.S. Pat. No. 6,084,325 discloses an elliptical trainer and a stationary bicycle which have a driving wheel assembly, a flywheel assembly, and a transmission belt assembly disposed therein, wherein the transmission belt assembly connects the driving wheel assembly and the flywheel assembly. The driving wheel assembly connects a crank and a pedal. When the user pedals the pedal, a driving wheel in the driving wheel assembly is driven to rotate, and the transmission belt assembly transmits the rotation force to the flywheel assembly, so as to drive a flywheel in the flywheel assembly to rotate. A plurality of permanent magnet are disposed on a peripheral edge of the flywheel. In addition, a fixed magnetic core is disposed inside of an elliptical trainer or a stationary bicycle, wherein the fixed magnetic core is electromagnetically coupled to the permanent magnets, and at least one set of coils surrounds the fixed magnetic core. When the flywheel rotates, the magnetic core and the rotating permanent magnets generate electromagnetic induction. In this way, the coil surrounding the fixed magnetic core generates an electric signal, and a control system of the fitness equipment performs feedback control according to the electric signal, whereby to generate resistance to the flywheel, allowing the user to adjust their exercise strength.

In order to enable users to not only adjust an exercise force by themselves but also monitor their exercise capacity, a measuring device is disposed inside the fitness equipment. However, the fitness equipment would be not only bulkier and occupies more space but also increases the manufacturing cost, which further increases the price of the fitness equipment and reduces the consumer's desire to purchase. Also, the accuracy of the measurement of the externally installed measuring device is skeptical.

Therefore, there is a need for the manufacturers to develop a measuring device with high accuracy which could occupy less space and have a lower cost.

### BRIEF SUMMARY OF THE INVENTION

In view of the above, the primary objective of the present invention is to provide a flywheel measuring system, which could measure a rider pedaling power outputted by the rider pedaling on the elliptical trainer and the stationary bicycle with high accuracy under a condition without over increasing the size and the cost of equipment.

The present invention provides a flywheel measuring system, which includes a base, a flywheel, at least one permanent magnet, a magnetic core, an abutting member, a cantilever arm, and a signal generating apparatus. The flywheel is rotatably disposed on the base, and has an axial core and a peripheral portion away from the axial core. The at least one permanent magnet is disposed on the peripheral portion. The magnetic core is movably disposed on the base and is electromagnetically coupled to the at least one permanent magnet, wherein when the flywheel rotates, the at least one permanent magnet drives the magnetic core to move. The abutting member is fixed on the base. The cantilever arm includes a connecting portion and a contacting portion which are disposed on the base, wherein when the magnetic core moves, the contacting portion is pushed by the magnetic core to abut against the abutting member. The signal generating apparatus is disposed on the contacting portion of the cantilever arm, wherein when the contacting portion abuts against the abutting member, the signal generating apparatus generates a strain, and outputs a signal according to the strain.

When the flywheel rotates, the permanent magnet disposed on the peripheral portion of the flywheel could move along with the rotation of the flywheel, and the permanent magnet could drive the magnetic core which is electromagnetically coupled to the permanent magnet to move. When the magnetic core moves to push the cantilever arm to approach the abutting member fixed on the base, the signal generating apparatus disposed on the cantilever arm would generate a strain, so as to generate a signal according to the strain.

In an embodiment, further including a supporting shaft and a restricting member, wherein the supporting shaft has a first end and a second end opposite to the first end; the first end is rotatably connected to the axial core of the flywheel, so that the supporting shaft rotates in a plane parallel to a rotation plane of the flywheel; the second end is connected to the magnetic core, so that the magnetic core rotates along a rotation direction of the supporting shaft; the restricting member is disposed on the base and is across the supporting shaft, whereby to restrict a rotation area of the supporting shaft.

In an embodiment, the base further comprises a recess, and the magnetic core further comprises a projecting portion disposed in the recess; when the magnetic core is driven by the at least one permanent magnet, the magnetic core moves along an extending direction of the recess.

In an embodiment, the signal generating apparatus is disposed between the contacting portion of the cantilever arm and the magnetic core.

In an embodiment, the signal generating apparatus is disposed between the contacting portion of the cantilever arm and the abutting member.

In an embodiment, the signal generating apparatus comprises a strain gauge.

In an embodiment, the signal generating apparatus comprises a piezoelectric material.

In an embodiment, the abutting member further includes a fixing portion and an abutting portion connected to the fixing portion; the fixing portion is fixed on the base; a distance between the abutting portion and the cantilever arm is adjustable by the abutting portion disposed on the fixing portion; when the magnetic core moves, the contacting portion of the cantilever arm is pushed by the magnetic core to approach the abutting portion.

In an embodiment, when the magnetic core is not moved, the magnetic core is in contact with the contacting portion, so that the contacting portion approaches the abutting member, and the signal generating apparatus generates an initial strain accordingly; when the magnetic core moves, the magnetic core pushes the contacting portion, so that the contacting portion is further close to the abutting member, and the signal generating apparatus further generates the strain from the initial strain and outputs a signal according to the strain.

In addition, the another primary objective of the present invention is to provide a fitness equipment, which could measure a rider pedaling power outputted by the rider with high accuracy under a condition without over increasing the size and the cost of equipment.

The present invention further provides a fitness equipment, which includes a base, a driving wheel assembly, a flywheel, a driven wheel assembly, at least one permanent magnet, a magnetic core, an abutting member, a cantilever arm, a signal generating apparatus, and a calculation module. The driving wheel assembly is disposed on the base and includes a crank assembly and a driving wheel connected to the crank assembly, wherein the crank assembly is adapted to be operated by a user to drive the driving wheel to rotate. The flywheel is rotatably disposed on the base and has an axial core and a peripheral portion away from the axial core. The driven wheel assembly is connected to the driving wheel and the flywheel and is adapted to drive the flywheel to rotate along with a rotation of the flywheel. The at least one permanent magnet is disposed on the peripheral portion of the flywheel. The magnetic core is movably disposed on the base and is electromagnetically coupled to the at least one permanent magnet, wherein when the flywheel rotates, the at least one permanent magnet drives the magnetic core to move. The abutting member is fixed on the base. The cantilever arm includes a connecting portion and a contacting portion which are disposed on the base, wherein when the magnetic core moves, the contacting portion is pushed by the magnetic core to abut against the abutting member. The signal generating apparatus is disposed on the contacting portion of the cantilever arm, wherein when the contacting portion abuts against the abutting member, the signal generating apparatus generates a strain, and outputs a signal according to the strain. The calculation module is connected to the signal generating apparatus for receiving the signal and is adapted to calculate a rider pedaling power outputted by the user according to the signal.

In an embodiment, when the magnetic core is not moved, the magnetic core is in contact with the contacting portion, so that the contacting portion approaches the abutting member, and the signal generating apparatus generates an initial strain accordingly; when the magnetic core moves, the magnetic core pushes the contacting portion, so that the contacting portion is further close to the abutting member, and the signal generating apparatus further generates the strain from the initial strain and outputs a signal according to the strain.

When the pedal of the fitness equipment is pedaled by a rider, the driving wheel of the driven wheel assembly is driven to rotate, so as to drive the flywheel to rotate via the driven wheel assembly. When the flywheel rotates, the permanent magnet disposed on the peripheral portion of the flywheel could move along with the rotation of the flywheel, and the permanent magnet could drive the magnetic core which is electromagnetically coupled to the permanent magnet to move. When the magnetic core moves to push the cantilever arm to approach the abutting member fixed on the base, the signal generating apparatus disposed on the cantilever arm would generate a strain, so as to generate a signal according to the strain. Then, the calculation module could calculate a power outputted by the rider pedaling on the pedal according to the signal.

With the aforementioned design, the disclosure of the present invention could measure a rider pedaling power outputted by the rider pedaling on the elliptical trainer and the stationary bicycle with high accuracy under a condition without over increasing the size and the cost of equipment.

### BRIEF DESCRIPTION OF THE SEVERAL VIEWS OF THE DRAWINGS

The present invention will be best understood by referring to the following detailed description of some illustrative embodiments in conjunction with the accompanying drawings, in which

FIG. 1 is a partial schematic diagram of the fitness equipment of an embodiment according to the present invention;

FIG. 2 is a schematic diagram of the fitness equipment, as seen from another direction of FIG. 1;

FIG. 3 is a schematic diagram of the fitness equipment, wherein a part of the components of the fitness equipment in FIG. 1 is omitted;

FIG. 4 is a partial schematic diagram of the fitness equipment of FIG. 3;

FIG. 5 is a partial schematic diagram of the fitness equipment of FIG. 3;

FIG. 6 is a schematic diagram, showing another type of the fitness equipment of FIG. 4;

FIG. 7 is a partial schematic diagram of the fitness equipment of another embodiment according to the present invention;

FIG. 8 is a partial schematic diagram of the fitness equipment of still another embodiment according to the present invention;

FIG. 9 is a partial schematic diagram of the fitness equipment of still another embodiment according to the present invention; and

FIG. 10 is a partial schematic diagram of the fitness equipment of FIG. 9.

### DETAILED DESCRIPTION OF THE INVENTION

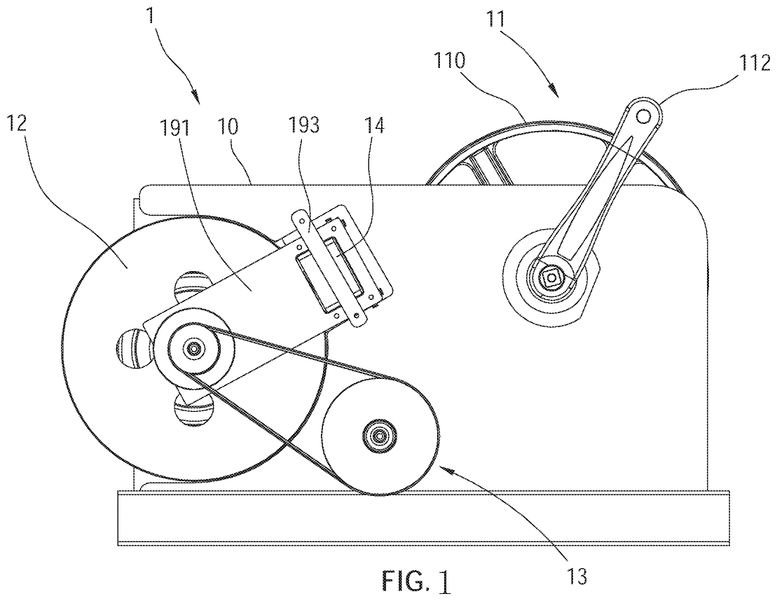

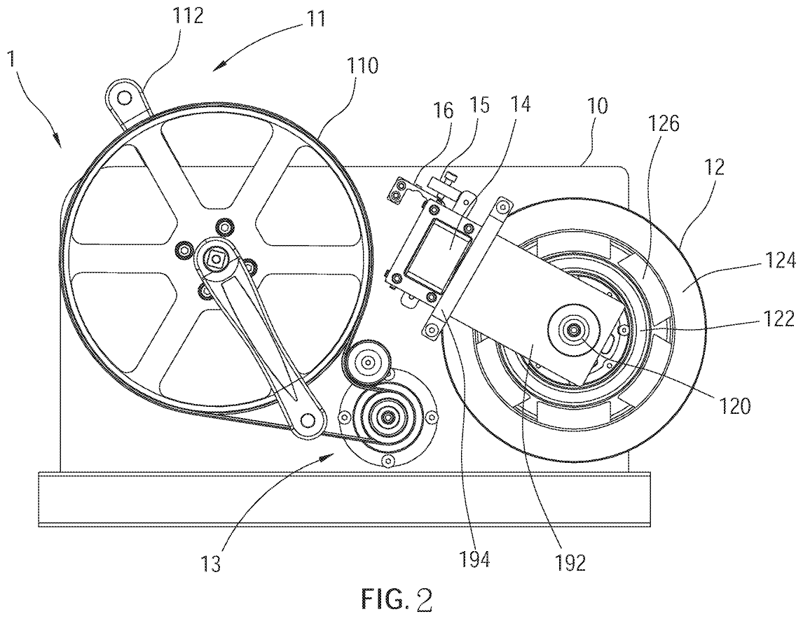

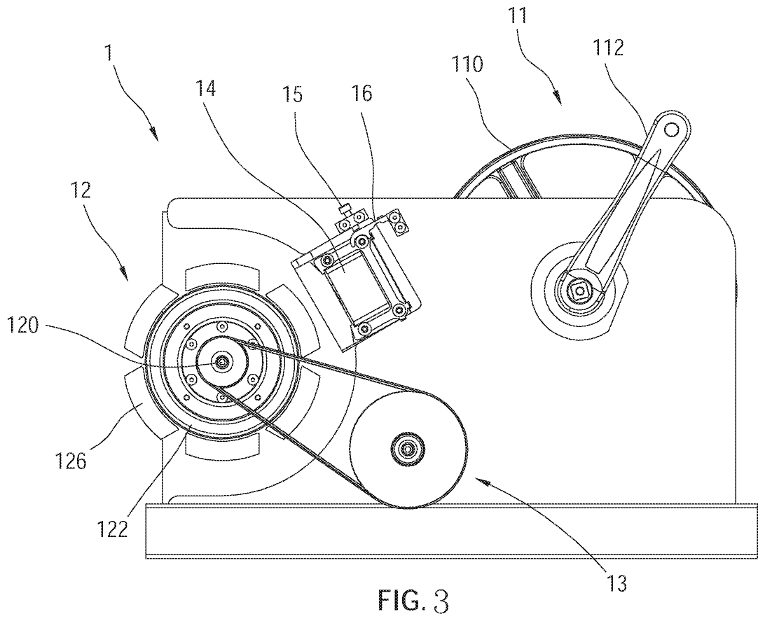

FIG. 1 to FIG. 3 are partial schematic diagrams of a fitness equipment 1 of an embodiment according to the present invention, wherein FIG. 2 is a schematic diagram of the fitness equipment 1, as seen from another direction of FIG. 1; FIG. 3 is a schematic diagram of the fitness equipment 1, wherein a part of the components of the fitness equipment 1 in FIG. 1 is omitted. As shown in FIG. 1, the fitness equipment 1 includes a base 10, a driving wheel assembly 11, a flywheel 12, and a driven wheel assembly 13, wherein the driving wheel assembly 11 includes a driving wheel 110 and a crank assembly 112 engaged with the driving wheel 110. The driving wheel 110 is rotatably disposed on the base 10. The crank assembly 112 is operable by a user to drive the driving wheel 110 to rotate. In practice, the fitness equipment 1 could be an elliptical trainer or a stationary bicycle, wherein the crank assembly 112 could be connected to a pedal of the fitness equipment 1, whereby the rider could pedal to rotate the driving wheel 110 via the crank assembly 112.

The driven wheel assembly 13 is connected to the driving wheel 110 and the flywheel 12, so that the rotation of the driving wheel 110 could be transmitted to the flywheel 12, and the flywheel 12 is driven to rotate correspondingly. The flywheel 12 includes an axial core 120, a peripheral portion 122 away from the axial core 120, and a casing 124. In addition, at least one permanent magnet 126 is disposed on the peripheral portion 122. In the current embodiment, the permanent magnet 126 is disposed between the peripheral portion 122 and the casing 124. In this way, when the flywheel 12 is driven to rotate, the permanent magnet 126 could move along with the rotation of the flywheel 12. In the current embodiment, the fitness equipment 1 includes six permanent magnets 126. However, the number of the permanent magnet 126 is not a limitation of the present invention. It is noted that the casing 124 is omitted in FIG. 3 in order to illustrate easily. In practice, the casing 124 would still participate in the operations and linkage of the fitness equipment 1.

Moreover, the fitness equipment 1 further includes a magnetic core 14, an abutting member 15, and a cantilever arm 16, wherein the magnetic core 14 is movably disposed on the base 10 and is electromagnetically coupled to the permanent magnet 126. In this way, when the flywheel 12 rotates, the permanent magnet 126 drives the magnetic core 14 to move. Both of the abutting member 15 and the cantilever arm 16 are disposed on the base 10. When the magnetic core 14 is driven by the permanent magnet 126, the cantilever arm 16 would be pushed to approach the abutting member 15.

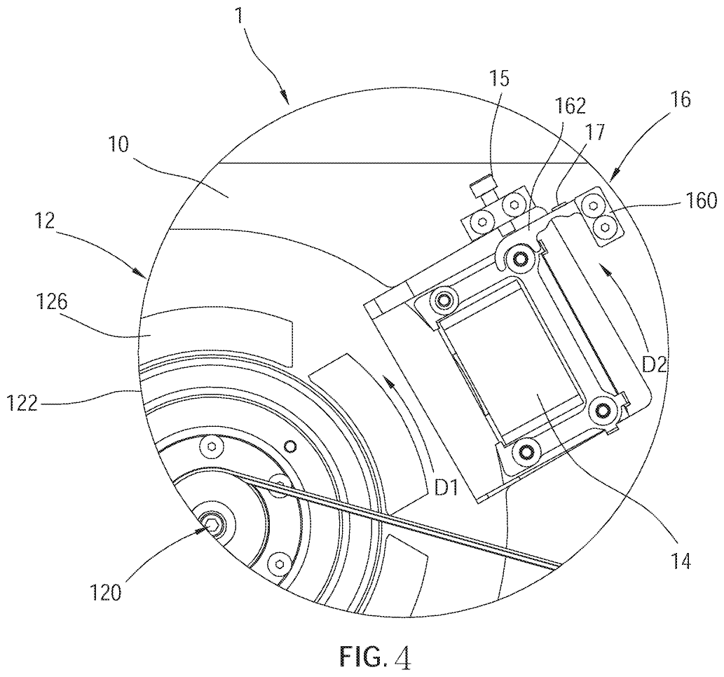

More specifically, as shown in FIG. 4 which is a partial schematic diagram of the fitness equipment 1 shown in FIG. 3, the cantilever arm 16 includes a connecting portion 160 and a contacting portion 162, wherein the connecting portion 160 is disposed on the base 10, and the contacting portion 162 is suspended on the base 10. In addition, the contacting portion 162 could be in contact with the magnetic core 14 and could approach the abutting member 15 by being pushed with the magnetic core 14. In practice, a projecting portion or a bolt on a housing of the magnetic core 14 could be adapted to contact the contacting portion 162. When the flywheel 12 is driven to rotate in a first direction D1, the permanent magnet 126 drives the magnetic core 14 to move in a second direction D2, so as to push the contacting portion 162 of the cantilever arm 16 to approach the abutting member 15. In the current embodiment, the fitness equipment 1 further includes a signal generating apparatus 17 disposed on the contacting portion 162 of the cantilever arm 16. When the magnetic core 14 is driven by the permanent magnet 126 to push the contacting portion 162 of the cantilever arm 16 to approach the abutting member 15, the signal generating apparatus 17 is pressed by the contacting portion 162 and the abutting member 15 to generate a strain, whereby to output a signal according to the strain. In the current embodiment, the signal generating apparatus 17 is a strain gauge. However, the signal generating apparatus 17 is not limited to be a strain gauge, but could be any apparatus or component (e.g. piezoelectric material) capable of converting the strain to signal to output. In practice, the abutting member 15 is adapted to limit the deformation of the contacting portion 162 of the cantilever arm 16, whereby to protect the cantilever arm 16.

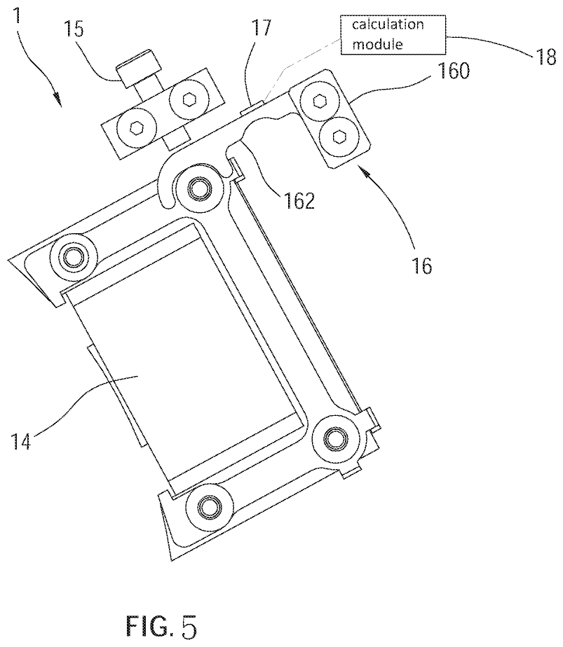

The signal generated by the signal generating apparatus 17 could be used to calculate an output power when the rider pedals the pedal. As shown in FIG. 5 which is a partial schematic diagram of the fitness equipment 1 of FIG. 3, the fitness equipment 1 further includes a calculation module 18 connected to the signal generating apparatus 17, wherein the calculation module 18 is adapted to receive the signal generated by the signal generating apparatus 17, whereby to calculate the output power when the rider pedals the pedal. More specifically, when the rider pedals faster, the strain generated by the signal generating apparatus 17 would increase and reflect in the output signal of the signal generating apparatus 17. In this way, the calculation module 18 could calculate a power outputted by the rider pedaling on the pedal according to the output signal of the signal generating apparatus 17. In practice, the calculation module 18 could display the calculated power on a display of the fitness equipment 1 for reference.

In conclusion, the permanent magnet 126, the magnetic core 14, the abutting member 15, the cantilever arm 16, and the signal generating apparatus 17, which are disposed on the base 10, constitute a flywheel measuring system. The signal outputted by the flywheel measuring system could be adapted to calculate a rider's pedaling power with high accuracy. In addition, as illustrated in the drawings, the flywheel measuring system occupies less space. Moreover, the cost of the flywheel measuring system is lower. Therefore, the fitness equipment 1 could measure an output power when the rider pedals the elliptical trainer or the stationary bicycle, etc., with high accuracy under a condition without over increasing the size and the cost of fitness equipment.

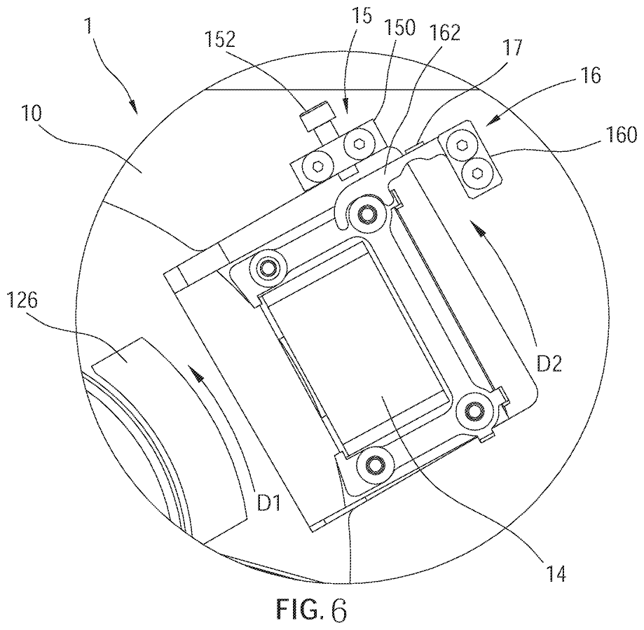

As shown in FIG. 6, which is a schematic diagram showing another type of the fitness equipment 1 in FIG. 4, the abutting member 15 of the fitness equipment 1 includes a fixing portion 150 and an abutting portion 152 engaged with the fixing portion 150, wherein the fixing portion 150 is fixed on the base 10, and the abutting portion 152 is disposed on the fixing portion 150 and could be adjusted to change a distance between the abutting portion 152 and the cantilever arm 16. In the current embodiment, the fixing portion 150 includes a threaded hole, and the abutting portion 152 is a bolt in conjunction with the threaded hole. As shown in FIG. 6, the abutting portion 152 could be adjusted upward to increase the distance between the abutting portion 152 and the contacting portion 162 of the cantilever arm 16. Alternatively, the abutting portion 152 could be adjusted downward to decrease the distance between the abutting portion 152 and the contacting portion 162. By adjusting the distance between the abutting portion 152 and the cantilever arm 16, the maximum deformation of the contacting portion 162 of the cantilever arm 16 could be changed, and the maximum strain generated by the signal generating apparatus 17 could be also changed.

As shown in FIG. 1 and FIG. 2, the fitness equipment 1 further includes two supporting shafts 191, 192, wherein an end of each of the supporting shafts 191, 192 is rotatably disposed on the axial core 120 of the flywheel 12 and is respectively located on two lateral sides of the flywheel 12, whereby each of the supporting shafts 191, 192 could rotate in a plane parallel to a rotation plane of the flywheel 12. It is noted that the supporting shafts 191, 192 are omitted in FIG. 3 in order to illustrate easily. In practice, the supporting shafts 191, 192 would still participate in operations and linkage of the fitness equipment 1. Another end of each of the supporting shafts 191, 192 is respectively connected to two lateral sides of the magnetic core 14. With the supporting shafts 191, 192, when the magnetic core 14 is driven by the permanent magnet 126, the magnetic core 14 could rotate along a rotation direction of the supporting shafts 191, 192. In addition, the fitness equipment 1 further includes two restricting members 193, 194 disposed on the base 10, wherein each of the restricting members 193, 194 is across one of the supporting shafts 191, 192. With the restricting members 193, 194, a rotation area of the supporting shafts 191, 192 is restricted. In other words, the movement of the magnetic core 14 is restricted too, whereby to protect the flywheel measuring system. For instance, when the pedal is pedaled under an excessive force, the permanent magnet 126 moving at a high speed may give the magnetic core 14 an excessive induction force, and make the magnetic core 14 be displaced in a wide range, so that the component is likely to break under an excessive pressure between the magnetic core 14, the cantilever arm 16, and the abutting member 15. However, the restricting members 193, 194 could restrict the movement of the magnetic core 14, preventing said problem. In addition, when the rider stops pedaling the fitness equipment 1 and the magnetic core 14 moves downward due to the gravity, the restricting members 193, 194 could be abutted by the magnetic core 14 as an original position, preventing the magnetic core 14 from falling out of the acting range.

However, the number of the supporting shaft is not limited to be two. In an embodiment, one supporting shaft and one restricting member are disposed on one side of the flywheel 12, whereby the movement of the magnetic core 14 could be restricted.

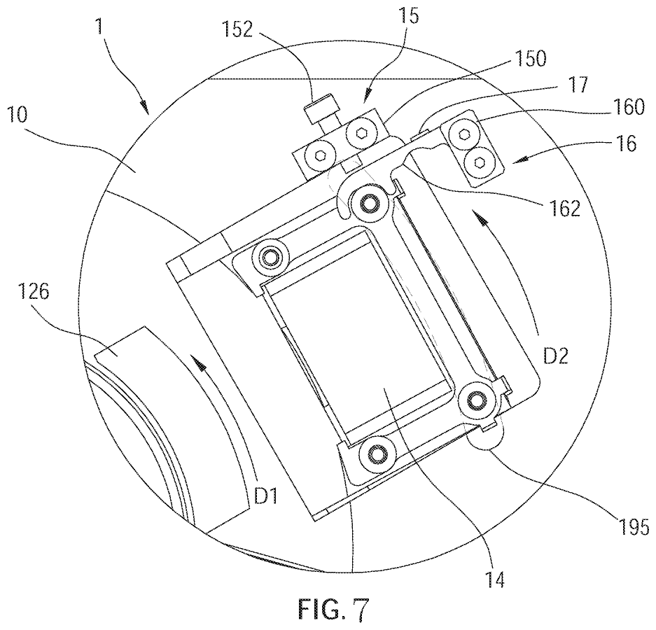

However, the restricting means that restricts the movement of the magnetic core 14 is not a limitation of the present invention. As shown in FIG. 1 and FIG. 7, wherein FIG. 7 is a partial schematic diagram of a fitness equipment 2 of another embodiment according to the present invention. The fitness equipment 2 has almost the same structure with the fitness equipment 1 in FIG. 1, except that the fitness equipment 2 of the current embodiment has a recess 195 which is adapted to receive a part of the magnetic core 14 (e.g. the projecting portion or a long bolt on the housing of the magnetic core 14). By disposing the projecting portion of the magnetic core 14 into the recess 195, the magnetic core 14 could move along an extending direction of the recess 195. In other words, the movement of the magnetic core 14 is restricted by the recess 195, which provides the same performance as the supporting shafts 191, 192 and the restricting members 193, 194. In practice, the fitness equipment could be provided with the recess 195, the supporting shafts 191, 192, and the restricting members 193, 194 at the same time for double protection

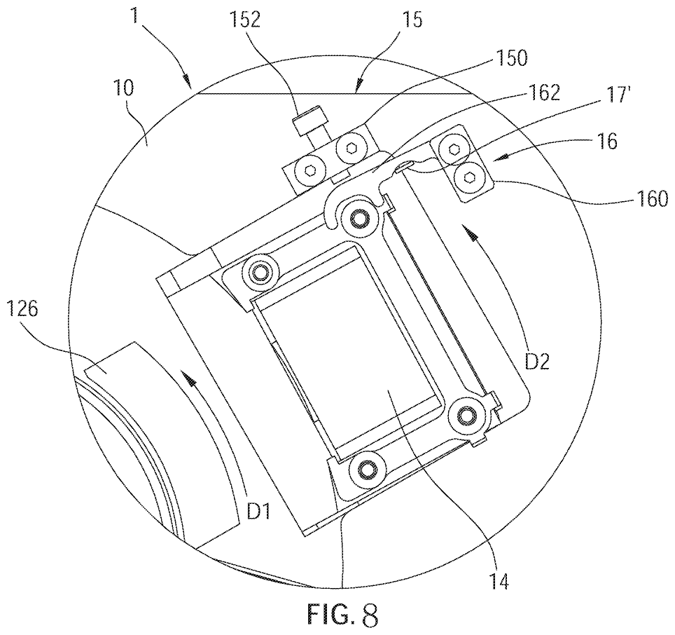

In the aforementioned embodiments, the signal generating apparatus 17 is disposed between the contacting portion 162 of the cantilever arm 16 and the abutting member 15 without being in contact with the abutting member 15. However, the position of the signal generating apparatus 17 is not a limitation of the present invention. As shown in FIG. 8 which is a partial schematic diagram of a fitness equipment 3 of still another embodiment according to the present invention, wherein the difference between the fitness equipment 3 and that of the aforementioned embodiments is that, a signal generating apparatus 17′ of the current embodiment is disposed on the contacting portion 162 of the cantilever arm 16 facing the magnetic core 14, preferably, the signal generating apparatus 17′ is located at a position that the signal generating apparatus 17′ would not interfere with other components. When the permanent magnet 126 drives the magnetic core 14 to move, the magnetic core 14 would be in contact with the signal generating apparatus 17′ to generate a strain, whereby to generate a signal according to the strain for the calculation module 18 to calculate an output power of the rider.

In the aforementioned embodiments, when the user uses the fitness equipment 1, the magnetic core 14 would be driven by the permanent magnet 126 to move upward, so as to push the contacting portion 162 of the cantilever arm 16 to approach the abutting member 15. In other words, the magnetic core 14 needs to overcome the force of gravity first to move upwards. It is necessary to consider the influence of gravity at the same time while calculating. However, the position of the magnetic core, the position of the cantilever arm, the position of the abutting member, and the position of the signal generating apparatus could be adjusted in other embodiments, whereby to reduce the influence of gravity.

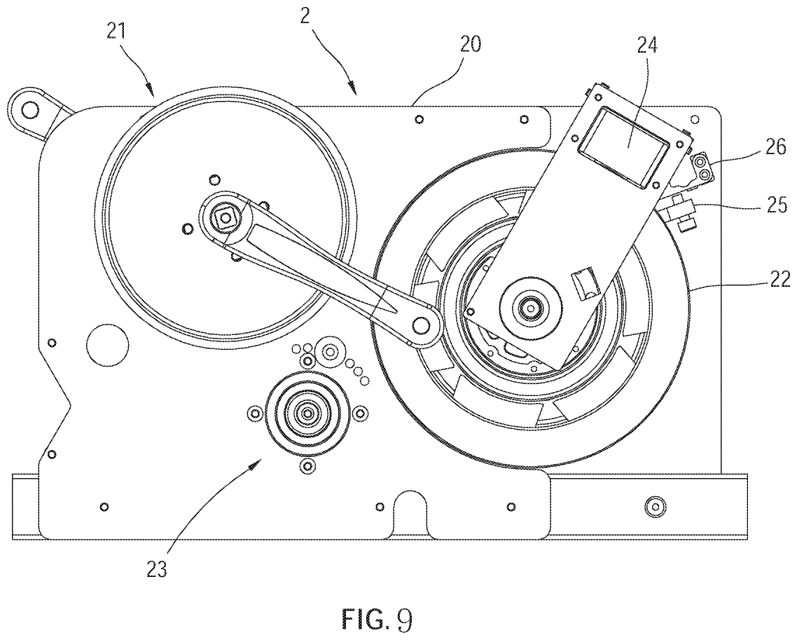

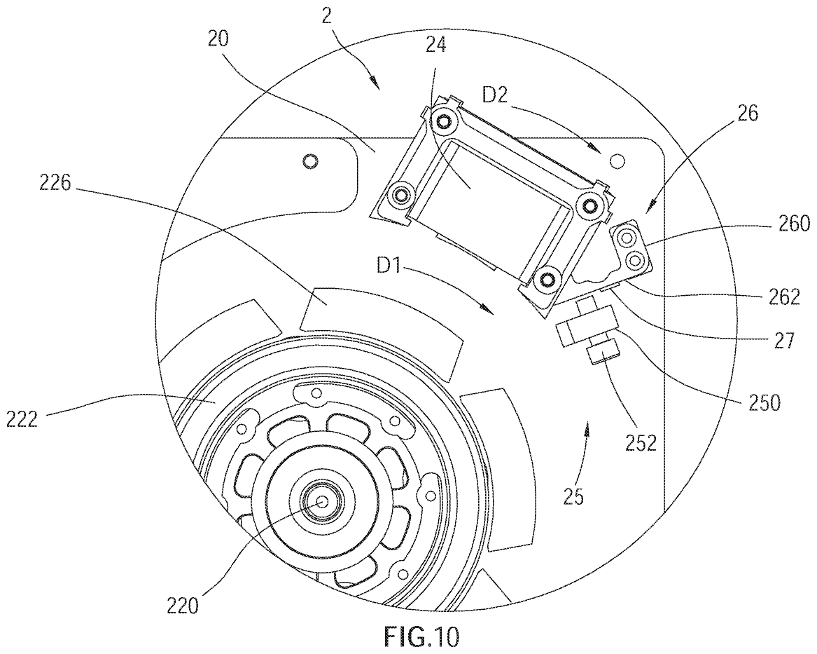

FIG. 9 is a partial schematic diagram showing still another type of a fitness equipment 2, and FIG. 10 is a partial schematic diagram of the fitness equipment 2 of FIG. 9. As shown in FIG. 9 and FIG. 10, the fitness equipment 2 includes a base 20, a driving wheel assembly 21, a flywheel 22, and a driven wheel assembly 23, which are substantially the same as that of the aforementioned embodiments, thus we are not going to describe in details herein. The difference between the fitness equipment 2 of the current embodiment and that of the aforementioned embodiments is that a magnetic core 24, an abutting member 25, a cantilever arm 26, and a signal generating apparatus 27 of the fitness equipment 2 of the current embodiment are disposed at different positions from that of the aforementioned embodiments. More specifically, the position of the magnetic core 24 could allow the magnetic core 24 to be moved downward with the rotation of a flywheel 22 of the fitness equipment 2 of the current embodiment. In the current embodiment, the cantilever arm 26 is disposed below the magnetic core 24, and the abutting member 25 is disposed below the cantilever arm 26, wherein the signal generating apparatus 27 is disposed between the cantilever arm 26 and the abutting member 25.

As shown in FIG. 10, in the current embodiment, when the rider stops pedaling the fitness equipment 2 and the magnetic core 24 abuts against a contacting portion 262 of the cantilever arm 26 due to the influence of gravity, the contacting portion 262 approaches an abutting portion 252 of the abutting member 25, and the signal generating apparatus 27 disposed between the cantilever arm 26 and the abutting member 25 thus generates an initial strain. Then, when the user uses the fitness equipment 2, the flywheel 22 and a permanent magnet 226 disposed thereon rotate in a direction D1, and the magnetic core 24 is driven to move in a direction D2 to push the contacting portion 262, so that the contacting portion 262 is further close to the abutting portion 252, causing the signal generating apparatus 27 to further generate additional strain from the initial strain. The signal generating apparatus 27 is capable of calculating an output power of the rider based on said additional strain. In this way, the disclosure of the present invention could reduce the influence of gravity and could measure the output power of the rider more precisely.

With the aforementioned embodiments, the flywheel measuring system of the present invention could measure a rider pedaling power outputted by the rider pedaling on the elliptical trainer and the stationary bicycle with high accuracy. In addition, the flywheel measuring system has a smaller volume, which would not excessively occupy the space of the fitness equipment. Moreover, the cost of the flywheel measuring system is lower, which would not excessively increase the price of the fitness equipment

It must be pointed out that the embodiments described above are only some preferred embodiments of the present invention. All equivalent structures which employ the concepts disclosed in this specification and the appended claims should fall within the scope of the present invention.

What is claimed is:

1. A flywheel measuring system, comprising:
a base;
a flywheel which is rotatably disposed on the base, wherein the flywheel has an axial core and a peripheral portion away from the axial core;
at least one permanent magnet disposed on the peripheral portion;
a magnetic core which is movably disposed on the base and is electromagnetically coupled to the at least one permanent magnet, wherein when the flywheel rotates, the at least one permanent magnet drives the magnetic core to move;
an abutting member fixed on the base;
a cantilever arm comprising a connecting portion and a contacting portion which are disposed on the base, wherein when the magnetic core moves, the contacting portion is pushed by the magnetic core to approach the abutting member; and
a signal generating apparatus disposed on the contacting portion of the cantilever arm, wherein when the contacting portion approaches the abutting member, the signal generating apparatus generates a strain, and outputs a signal according to the strain.

2. The flywheel measuring system of claim 1, further comprising a supporting shaft and a restricting member, wherein the supporting shaft has a first end and a second end opposite to the first end; the first end is rotatably connected to the axial core of the flywheel, so that the supporting shaft rotates in a plane parallel to a rotation plane of the flywheel; the second end is connected to the magnetic core, so that the magnetic core rotates along a rotation direction of the supporting shaft; the restricting member is disposed on the base and is across the supporting shaft, whereby to restrict a rotation area of the supporting shaft.

3. The flywheel measuring system of claim 1, wherein the base further comprises a recess, and the magnetic core further comprises a projecting portion disposed in the recess; when the magnetic core is driven by the at least one permanent magnet, the magnetic core moves along an extending direction of the recess.

4. The flywheel measuring system of claim 1, wherein the signal generating apparatus is disposed between the contacting portion of the cantilever arm and the magnetic core.

5. The flywheel measuring system of claim 1, wherein the signal generating apparatus is disposed between the contacting portion of the cantilever arm and the abutting member.

6. The flywheel measuring system of claim 1, wherein the signal generating apparatus comprises a strain gauge.

7. The flywheel measuring system of claim 1, wherein the signal generating apparatus comprises a piezoelectric material.

8. The flywheel measuring system of claim 1, wherein the abutting member further comprises a fixing portion and an abutting portion connected to the fixing portion; the fixing portion is fixed on the base; a distance between the abutting portion and the cantilever arm is adjustable by the abutting portion disposed on the fixing portion; when the magnetic core moves, the contacting portion of the cantilever arm is pushed by the magnetic core to approach the abutting portion.

9. The flywheel measuring system of claim 1, wherein when the magnetic core is not moved, the magnetic core is in contact with the contacting portion, so that the contacting portion approaches the abutting member, and the signal generating apparatus generates an initial strain accordingly; when the magnetic core moves, the magnetic core pushes the contacting portion, so that the contacting portion is further close to the abutting member, and the signal generating apparatus further generates the strain from the initial strain and outputs a signal according to the strain.

10. A fitness equipment, comprising:
a base;
a driving wheel assembly disposed on the base; wherein the driving wheel assembly comprises a crank assembly and a driving wheel connected to the crank assembly; the crank assembly is adapted to be operated by a user to drive the driving wheel to rotate;
a flywheel which is rotatably disposed on the base and has an axial core and a peripheral portion away from the axial core;
a driven wheel assembly which is connected to the driving wheel and the flywheel and is adapted to drive the flywheel to rotate along with a rotation of the flywheel;
at least one permanent magnet disposed on the peripheral portion of the flywheel;
a magnetic core which is movably disposed on the base and is electromagnetically coupled to the at least one permanent magnet, wherein when the flywheel rotates, the at least one permanent magnet drives the magnetic core to move;
an abutting member fixed on the base;
a cantilever arm comprising a connecting portion and a contacting portion which are disposed on the base, wherein when the magnetic core moves, the contacting portion is pushed by the magnetic core to approach the abutting member;
a signal generating apparatus disposed on the contacting portion of the cantilever arm, wherein when the contacting portion approach the abutting member, the signal generating apparatus generates a strain, and outputs a signal according to the strain; and
a calculation module which is connected to the signal generating apparatus for receiving the signal and is adapted to calculate a rider pedaling power outputted by the user according to the signal.

11. The fitness equipment of claim 10, wherein when the magnetic core is not moved, the magnetic core is in contact with the contacting portion, so that the contacting portion approaches the abutting member, and the signal generating apparatus generates an initial strain accordingly; when the magnetic core moves, the magnetic core pushes the contacting portion, so that the contacting portion is further close to the abutting member, and the signal generating apparatus further generates the strain from the initial strain and outputs a signal according to the strain.
